(19) United States
(12) Patent Application Publication
(10) Pub. No.: US 2022/0027535 A1
(43) Pub. Date: Jan. 27, 2022

(54) INFORMATION PROCESSING APPARATUS, COSMETIC GENERATOR, AND COMPUTER PROGRAM

(71) Applicant: SHISEIDO COMPANY, LTD., Tokyo (JP)

(72) Inventors: Naruhito TOYODA, Tokyo (JP); Megumi SEKINO, Tokyo (JP); Kanako KANEKO, Tokyo (JP)

(73) Assignee: SHISEIDO COMPANY, LTD., Tokyo (JP)

(21) Appl. No.: 17/296,271

(22) PCT Filed: Nov. 1, 2019
(86) PCT No.: PCT/JP2019/043133

§ 371 Date: May 24, 2021

(51) Int. Cl. G06F 30/27; G16H 50/30; A45D 44/00; A45D 40/24

(52) U.S. Cl. CPC G06F 30/27; G16H 50/30; A45D 2034/005; A45D 40/24; A45D 2044/007; A45D 44/005

(57) ABSTRACT

An information processing apparatus that executes a simulation that predicts future skin condition when cosmetics are used on the skin. The apparatus retrieves target person skin information regarding simulation target person's skin, predicts transition of skin condition of the simulation target person by inputting the target person's skin information to condition transition model relating to the skin condition transition based on subject skin information indicating the time course of the skin when each of the plurality of subjects uses the cosmetic on the skin, subject condition information regarding the subject's skin condition corresponding to each subject's skin information, and cosmetic information regarding the cosmetic, and presents the predicted skin condition transition.

### TECHNICAL FIELD

The present invention relates to an information processing apparatus, a cosmetic generator, and a computer program.

### BACKGROUND ART

In general, it is important for a cosmetic consumer selecting a cosmetic to know change in skin condition obtained by the cosmetic.

It is known that the skin condition can be predicted from subcutaneous tissue of the skin.

For example, Japanese Patent Application Laid-Open No. 2014-064949 discloses a technique for estimating the internal state of the skin based on light reflection characteristics of the subcutaneous tissue of the skin.

### SUMMARY OF INVENTION

### Technical Problem

In reality, the change in skin condition obtained by cosmetics is not determined by the time when the cosmetic is used once, but by the duration of utilization of the cosmetic, the frequency of use, and the usage amount used per use.

However, in Japanese Patent Application Laid-Open No. 2014-064949, the skin condition is estimated based on the condition of the subcutaneous tissue when the cosmetic is used once, so that the estimation result indicates the skin condition at the time when the cosmetic is used. It does not indicate the future skin condition when the utilization of cosmetics is continued.

An object of the present invention is to predict future skin condition when the utilization of cosmetics is continued.

### Solution to Problem

One aspect of the present invention is an information processing apparatus that executes a simulation that predicts future skin condition when cosmetics are used on the skin, the apparatus:

retrieving target person skin information regarding simulation target person's skin;

predicting transition of skin condition of the simulation target person by inputting the target person's skin information to condition transition model relating to the skin condition transition based on subject skin information indicating the time course of the skin when each of the plurality of subjects uses the cosmetic on the skin, subject condition information regarding the subject's skin condition corresponding to each subject's skin information, and cosmetic information regarding the cosmetic; and

presenting the predicted skin condition transition.

### Advantageous Effects of Invention

According to the present invention, it is possible to predict future skin condition when the utilization of cosmetics is continued.

### BRIEF DESCRIPTION OF DRAWINGS

FIG. 1 is a block diagram showing a configuration of an information processing system according to the present embodiment.

FIG. 2 is a block diagram showing a configuration of the cosmetic generator 50 of FIG. 1.

FIG. 3 is an explanatory diagram of summary of the present embodiment.

FIG. 4 is a diagram showing a data structure of a user information database of the present embodiment.

FIG. 5 is a diagram showing a data structure of a subject information database of the present embodiment.

FIG. 6 is a diagram showing a data structure of a cosmetic information master database of the present embodiment.

FIG. 7 is a diagram showing a data structure of a class information master database of the present embodiment.

FIG. 8 is a conceptual diagram of a condition transition model corresponding to the class information master database of FIG. 7.

FIG. 9 is a conceptual diagram showing inputs and outputs of the condition transition model of FIG. 8.

FIG. 10 is a diagram showing a data structure of a simulation log information database of the present embodiment.

FIG. 11 is a sequence diagram of a simulation process of the present embodiment.

FIG. 12 is a diagram showing an example of a screen displayed in the information processing of FIG. 11.

FIG. 13 is a diagram showing an example of a screen displayed in the information processing of the first variation.

FIG. 14 is a flowchart of information processing of the second variation.

FIG. 15 is an explanatory diagram of summary of the third variation.

FIG. 16 is a diagram showing a data structure of community information database of the third variation.

FIG. 17 is a sequence diagram of a community analysis process of the third variation.

### DESCRIPTION OF EMBODIMENTS

Hereinafter, an embodiment of the present invention will be described in detail based on the drawings.

Note that, in the drawings for describing the embodiments, the same components are denoted by the same reference sign in principle, and the repetitive description thereof is omitted.

### (1) Configuration of Information Processing System

The configuration of an information processing system will be described.

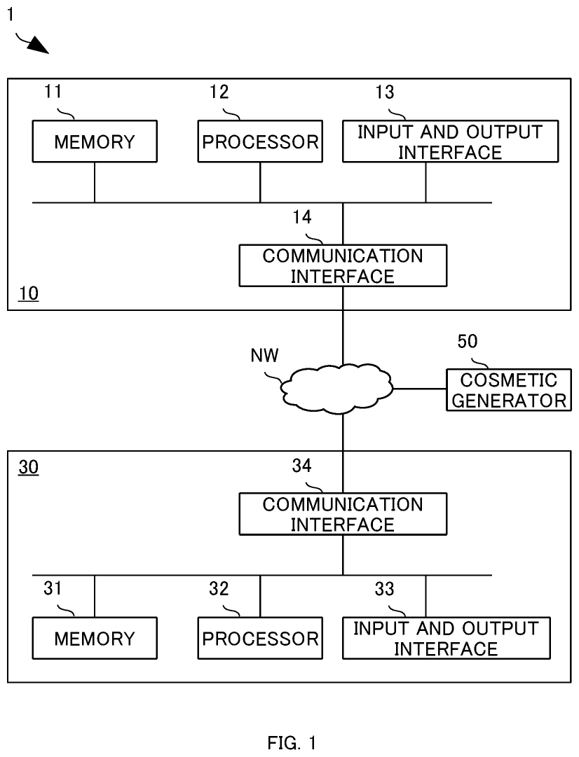

FIG. 1 is a block diagram showing the configuration of the information processing system according to the present embodiment.

As shown in FIG. 1, the information processing system 1 includes a client apparatus 10, a server 30, and a cosmetic generator 50.

The client apparatus 10, the server 30, and the cosmetic generator 50 are connected via a network (for example, the Internet or an intranet) NW.

The client apparatus 10 is an example of an information processing apparatus that transmits a request to the server 30.

The client apparatus 10 is, for example, a smartphone, a tablet terminal, or a personal computer.

The server 30 is an example of an information processing apparatus that provides the client apparatus 10 with a response in response to the request transmitted from the client apparatus 10.

The server 30 is, for example, a web server.

The cosmetic generator 50 is configured to generate cosmetics based on the cosmetic information transmitted from the client apparatus 10 or the server 30.

The cosmetics are, for example, at least one of the following:

skin care cosmetics (for example, at least one of lotion, milky lotion, serum, facial cleanser, cream, and facial mask); and

makeup cosmetics (for example, at least one of makeup base, foundation, concealer, facial powder, lipstick, lip gloss, eye shadow, eyeliner, mascara, eyelash, teak, and coffret).

### (1-1) Configuration of Client Apparatus

The configuration of the client apparatus 10 will be described with reference to FIG. 1.

As shown in FIG. 1, the client apparatus 10 includes a memory 11, a processor 12, an input and output interface 13, and a communication interface 14.

The memory 11 is configured to store a program and data.

The memory 11 is, for example, a combination of a ROM (read only memory), a RAM (random access memory), and a storage (for example, a flash memory or a hard disk).

The program includes, for example, the following program.

OS (Operating System) program; and

application program (for example, web browser) for executing information processing.

The data includes, for example, the following data:

database referenced in information processing; and

data obtained by executing an information processing (that is, an execution result of an information processing)

The processor 12 is configured to realize the function of the client apparatus 10 by activating the program stored in the memory 11.

The processor 12 is an example of a computer.

The input and output interface 13 is configured to retrieve a user's instruction from an input apparatus connected to the client apparatus 10 and output information to an output apparatus connected to the client apparatus 10.

The input device is, for example, a keyboard, a pointing device, a touch panel, or a combination thereof.

The output device is, for example, a display.

The communication interface 14 is configured to control communication via the network NW.

### (1-2) Server Configuration

The configuration of the server 30 will be described with reference to FIG. 1.

As shown in FIG. 1, the server 30 includes a memory 31, a processor 32, an input and output interface 33, and a communication interface 34.

The memory 31 is configured to store a program and data.

The memory 31 is, for example, a combination of ROM, RAM, and storage (for example, flash memory or hard disk).

The program includes, for example, the following program:

OS program; and

application program for executing information processing.

The data includes, for example, the following data:

database referenced in information processing; and

execution result of information processing

The processor 32 is configured to realize the function of the server 30 by activating the program stored in the memory 31.

The processor 32 is an example of a computer.

The input and output interface 33 is configured to retrieve a user's instruction from an input apparatus connected to the server 30 and output information to an output apparatus connected to the server 30.

The input device is, for example, a keyboard, a pointing device, a touch panel, or a combination thereof.

The output device is, for example, a display.

The communication interface 34 is configured to control communication via the network NW.

### (1-3) Configuration of Cosmetic Generator

The configuration of the cosmetic generator 50 of the present embodiment will be described.

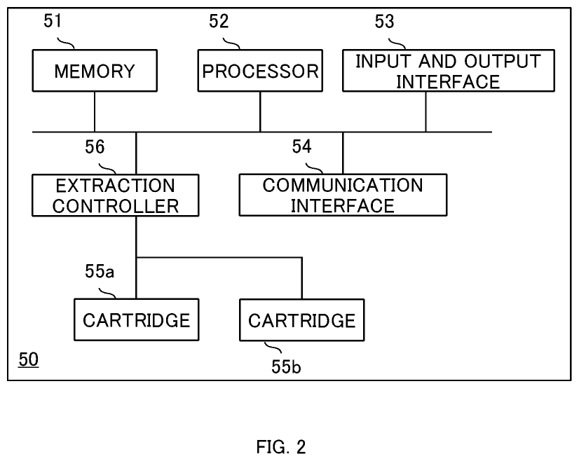

FIG. 2 is a block diagram showing the configuration of the cosmetic generator 50 of FIG. 1.

As shown in FIG. 2, the cosmetic generator 50 includes a memory 51, a processor 52, an input and output interface 53, a communication interface 54, an extraction controller 56, and a plurality of cartridges 55a to 55b.

The memory 51 is configured to store a program and data.

The memory 51 is, for example, a combination of ROM, RAM, and storage (for example, flash memory or hard disk).

The program includes, for example, the following program:

OS program; and

control program for the cosmetic generator 50

The data includes, for example, the following data:

information on history of cosmetic generation;

information on the remaining amount of raw materials contained in a plurality of cartridges 55a to 55b.

The processor 52 is configured to realize the function of the cosmetic generator 50 by activating the program stored in the memory 51.

The processor 52 is an example of a computer.

The input and output interface 53 is configured to retrieve a user's instruction from an input apparatus connected to the cosmetic generator 50 and output information to an output apparatus connected to the cosmetic generator 50.

The input device is, for example, a keyboard, a pointing device, a touch panel, or a combination thereof.

The output device is, for example, a display.

The communication interface 54 is configured to control communication via the network NW.

Raw materials for cosmetics are stored in each of the cartridges 55a to 55b.

The extraction controller 56 is configured to extract raw materials from each of the cartridges 55a to 55b based on the cosmetic information transmitted from the client apparatus 10 or the server 30.

### (2) Summary of the Embodiment

The summary of the present embodiment will be described.

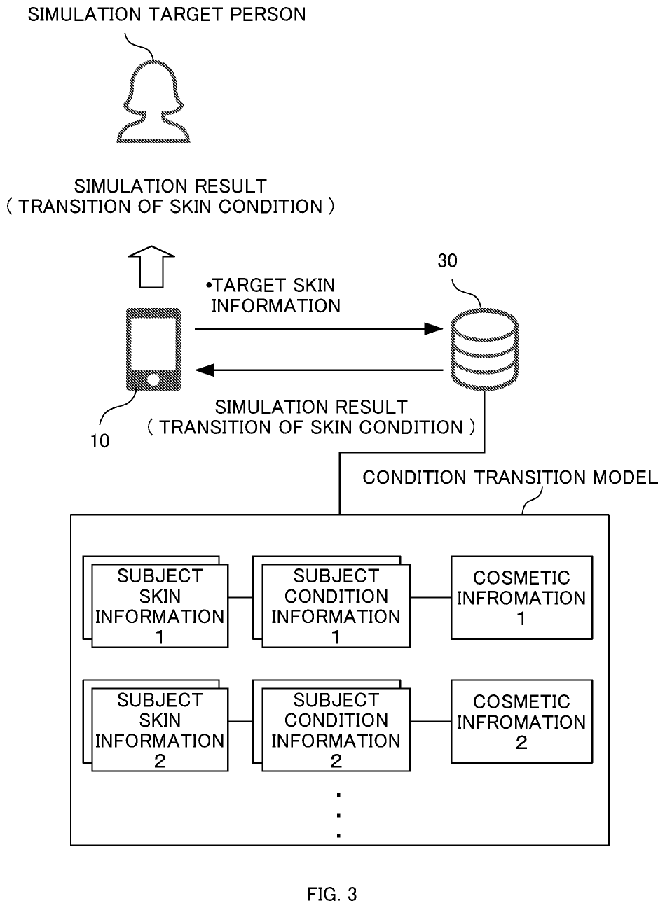

FIG. 3 is an explanatory diagram of a summary of the present embodiment.

As shown in FIG. 3, the server 30 is configured to execute a simulation of the skin condition when the cosmetic is used to the skin.

The server 30 generates a condition transition model related to transition of the skin condition based on a plurality of subject skin information indicating the time course of the skin when each of the plurality of subjects uses the cosmetic on the skin, and subject skin condition information regarding the subject's skin condition corresponding to each subject's skin information, and cosmetic information regarding cosmetics.

The server 30 retrieves cosmetic information regarding the target cosmetic to be simulated via the client apparatus 10.

The server 30 retrieves the target person skin information of the simulation target person via the client apparatus 10.

The server 30 predicts the transition of the skin condition of the simulation target person by inputting the cosmetic information of the target cosmetic and the skin information of the target person into the condition transition model.

The server 30 presents the predicted transition of the skin condition via the client apparatus 10.

According to this embodiment, it is possible to predict the future skin condition when the utilization of the cosmetic is continued.

### (3) Database

The database of the present embodiment will be described.

The following database is stored in the memory 31.

### (3-1) User Information Database

The user information database of the present embodiment will be described.

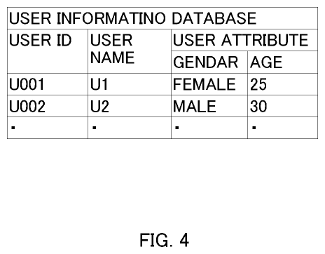

FIG. 4 is a diagram showing a data structure of the user information database of the present embodiment.

The user information database of FIG. 4 stores user information regarding the user.

The user information database includes a “user ID” field, a “user name” field, and a “user attribute” field.

Each field is associated with each other.

The “user ID” field stores a user ID.

The user ID is an example of user identification information that identifies a user.

The Username field stores information regarding the username (for example, text).

The “user attribute” field stores user attribute information regarding the user's attribute.

The “user attribute” field includes a plurality of subfields (“gender” field and “age” field).

The “gender” field stores information regarding the user's gender.

The “age” field stores information regarding the age of the user.

### (3-2) Subject Information Database

The subject information database of the present embodiment will be described.

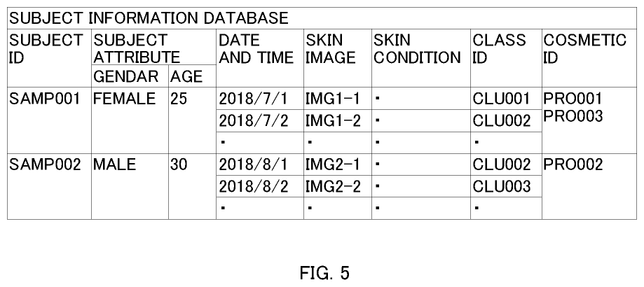

FIG. 5 is a diagram showing a data structure of the subject information database of the present embodiment.

The subject information database of FIG. 5 stores subject information regarding the subject.

The subject information database includes a “subject ID” field, a “subject attribute” field, a “date and time” field, a “skin image” field, a “skin condition” field, a “class ID” field, and a “cosmetic ID”.

Each field is associated with each other.

The “subject ID” field stores subject ID.

The subject ID is an example of subject identification information that identifies a subject.

The “subject attribute” field stores subject attribute information regarding the subject's attributes.

The “subject attributes” field includes a plurality of subfields (“gender” field and “age” field).

The “gender” field stores information regarding the subject's gender.

The “age” field stores information regarding the age of the subject.

The “date and time” field stores information regarding the date and time when the measurement was performed on the subject.

The “skin image” field stores image data regarding the subject's skin.

The “skin condition” field stores skin condition information (an example of “subject condition information”) regarding the skin condition determined based on the image data of the skin of the subject.

The “date and time” field, the “skin image” field, and the “skin condition” field are associated with each other.

Records are added to the “date and time” field, “skin image” field, and “skin condition” field each time a measurement is performed.

That is, in the “skin image” field stores image data showing the time course of the skin when the subject uses the cosmetic on the skin.

The “skin condition” field stores information indicating the time course of the skin condition when the subject uses the cosmetic on the skin.

The “class ID” field stores a class ID (an example of “class identification information”) that identifies a class corresponding to the skin image data (that is, a class to which the skin condition determined from the feature quantity of the skin image data belongs).

The “cosmetic ID” field stores a cosmetic ID (an example of “makeup identification information”) that identifies the makeup used by the subject.

### (3-3) Cosmetic Information Master Database

The cosmetic information master database of the present embodiment will be described.

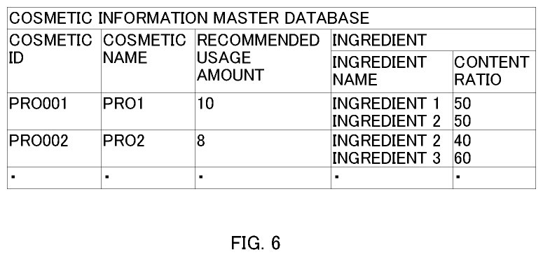

FIG. 6 is a diagram showing a data structure of the cosmetic information master database of the present embodiment.

The cosmetic information master database of FIG. 6 stores cosmetic information regarding cosmetics.

The cosmetic information master database includes a “cosmetic ID” field, a “cosmetic name” field, a “recommended usage amount” field, and an “ingredient” field.

Each field is associated with each other.

“cosmetic ID” field stores cosmetic ID.

The “cosmetic name” field stores information (for example, text) regarding the cosmetic name.

The “recommended usage amount” field stores information regarding the recommended usage amount of cosmetics.

The “ingredients” field stores information regarding the ingredients of the cosmetic.

The “ingredient” field includes a plurality of subfields (“ingredient name” field and “content ratio” field).

The “ingredient name” field stores information regarding the name of the ingredient of the cosmetic (for example, a text or an ingredient code for identifying the ingredient).

The “content ratio” field stores information regarding the content ratio of each component.

### (3-4) Class Information Master Database

The class information master database of the present embodiment will be described.

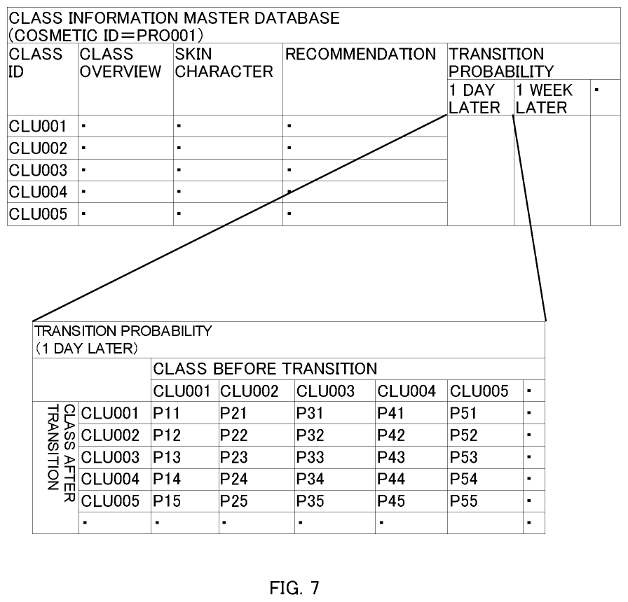

FIG. 7 is a diagram showing a data structure of the class information master database of the present embodiment.

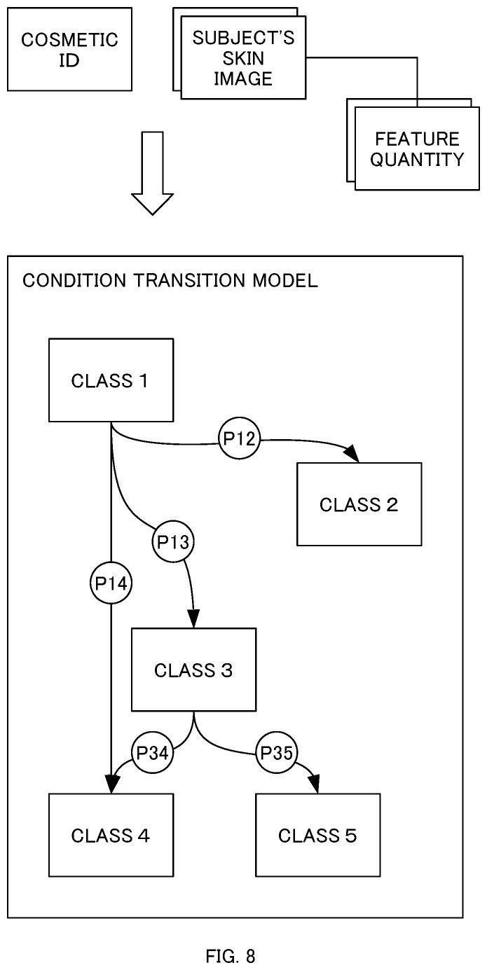

FIG. 8 is a conceptual diagram of a condition transition model corresponding to the class information master database of FIG. 7.

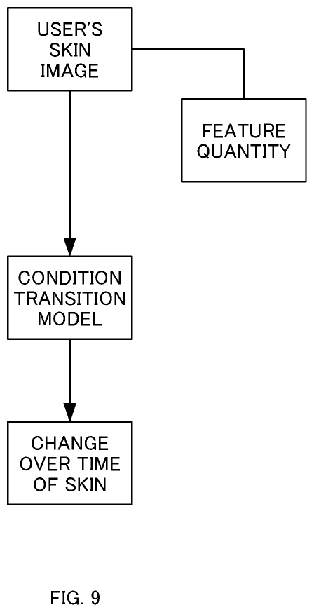

FIG. 9 is a conceptual diagram showing the input and output of the condition transition model of FIG. 8.

The class information master database of FIG. 7 stores class information.

The class information is information regarding the classification of skin based on the feature quantity of the skin image data of a plurality of subjects.

The class information master database includes a “class ID” field, a “class overview” field, a “skin character” field, a “recommendation” field, and a “transition probability” field.

Each field is associated with each other.

The class information master database is associated with the cosmetic ID.

The “class ID” field stores class ID.

The “class summary” field stores information (for example, text) regarding a class summary description.

The information in the “class summary” field is determined by the administrator of the server 30.

The “skin character” field stores skin character information regarding skin characters corresponding to each class.

The skin character information shows at least one of the following:

characteristics of skin texture (for example, texture flow and texture distribution range);

characteristics of the skin groove (for example, the state of the skin groove and the shape of the skin groove);

characteristics of pores (for example, condition of pores, size of pores, number of pores, and density of pores); and

characteristics of skin hills (for example, size of skin hills, number of skin hills, density of skin hills).

The “recommendation” field stores the recommendation information corresponding to each class.

The recommendation information includes the recommendation information regarding skin care and the recommendation information regarding makeup.

Recommendation information regarding skin care includes, for example, at least one of the following:

types of makeup base;

types of foundation;

coating means (for example, figure coating or sponge coating);

coating method;

types of cosmetics; and

usage amount of cosmetics (for example, light coating or thick coating)

Recommendation information regarding makeup includes, for example, at least one of the following:

recommended skin care methods (for example, daily care methods using lotion or milky lotion, and special care methods using beauty essence, cream or mask);

use method of utilization cosmetics in utilization (as an example, frequency of utilization and usage amount at one time);

cosmetics recommended for utilization (hereinafter referred to as “recommended cosmetic”); and

recommended usage amount of cosmetics (as an example, frequency of utilization and usage amount at one time).

The “transition probability” field contains a plurality of subfields.

The plurality of subfields includes each future time point (for example, 1 day, 1 week, 1 month, 3 months, 6 months, and 1 year).

Each subfield is assigned to class ID and stores a transition probability between classes.

In the example of FIG. 7, among the “transition probability” fields of the record in which the class ID “CLU001” is stored in the “class ID” field, the subfield to which the future time point “1 day later” is assigned stores the transition probabilities P11 to P15 one day after the class of the class ID “CLU001”.

Among the “transition probability” fields of the record in which the class ID “CLU002” is stored in the “class ID” field, the subfield to which the future time point “1 day later” is assigned stores the transition probabilities P21 to P25 after one day from the class of the class ID “CLU002”.

Among the “transition probability” fields of the record in which the class ID “CLU001” is stored in the “class ID” field, the subfield to which the future time point “1 year later” is assigned stores the transition probability after one year from the class of the class ID “CLU001”.

The condition transition model of FIG. 8 corresponds to the class information master database of FIG. 7.

That is, the condition transition model is associated with cosmetic ID and is generated based on skin image data (that is, changes over time of the skin) of each of a plurality of subjects using cosmetic associated with the cosmetic ID.

The condition transition model includes a plurality of classes and links between each class.

The link shows the probability of transition between each class.

As an example, the condition transition model of FIG. 8 includes class 1 of class ID “CLU001” to class 5 of class ID “CLU005”.

The links between each class are as follows:

transition probability from class 1 to class 2 after 1 day: P12;

transition probability from class 1 to class 3 after 1 day: P13;

transition probability from class 1 to class 4 after 1 day: P14;

transition probability from class 2 to class 5 after 1 day: P25;

transition probability from class 3 to class 4 after 1 day: P34; and

transition probability from class 3 to class 5 after 1 day: P35.

As shown in FIG. 9, the input of the condition transition model is image data.

The output of the condition transition model is the prediction result of the time-dependent change of the skin condition when the cosmetic corresponding to the cosmetic ID is used.

The class information master database (FIG. 7) and the condition transition model (FIG. 8) are generated by any of the following methods:

unsupervised learning;

supervised learning; and

network analysis.

### (3-5) Simulation Log Information Database

The simulation log information database of the present embodiment will be described.

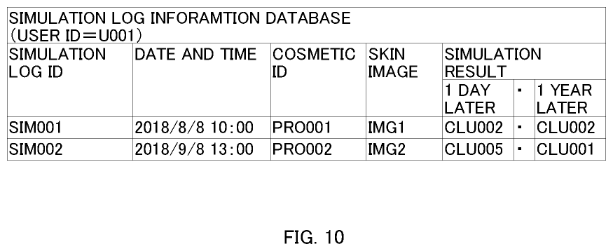

FIG. 10 is a diagram showing a data structure of the simulation log information database of the present embodiment.

The simulation log information database of FIG. 10 stores simulation log information regarding the history of simulation execution results.

The simulation log information database includes a “simulation log ID” field, a “date and time” field, a “cosmetic ID” field, a “skin image” field, and a “simulation result” field.

Each field is associated with each other.

The simulation log information database is associated with the user ID.

The “Simulation log ID” field stores simulation log ID that identifies simulation log information.

The “date and time” field stores information regarding the simulation execution date and time.

The “cosmetic ID” field stores the cosmetic ID of the cosmetic to be simulated.

The “skin image” field stores image data of the skin image to be simulated.

The “simulation result” field stores information regarding the execution result of the simulation.

The “simulation results” fields include “1 day later” field, “1 week later” field, “1 month later” field, “3 months later” field, “6 months later” field, and “1 year later” field.

The “1 day later” field stores the class ID of the class to which the skin belongs 1 day after the execution date and time of the simulation.

The “1 week later” field stores the class ID of the class to which the skin belongs one week after the execution date and time of the simulation.

The “1 month later” field stores the class ID of the class to which the skin belongs 1 month after the execution date and time of the simulation.

The “3 months later” field stores the class ID of the class to which the skin belongs 3 months after the execution date and time of the simulation.

The “6 months later” field stores the class ID of the class to which the skin belongs 6 months after the execution date and time of the simulation.

The “1 year later” field stores the class ID of the class to which the skin belongs one year after the execution date and time of the simulation.

### (4) Information Processing

Information processing of the present embodiment will be described.

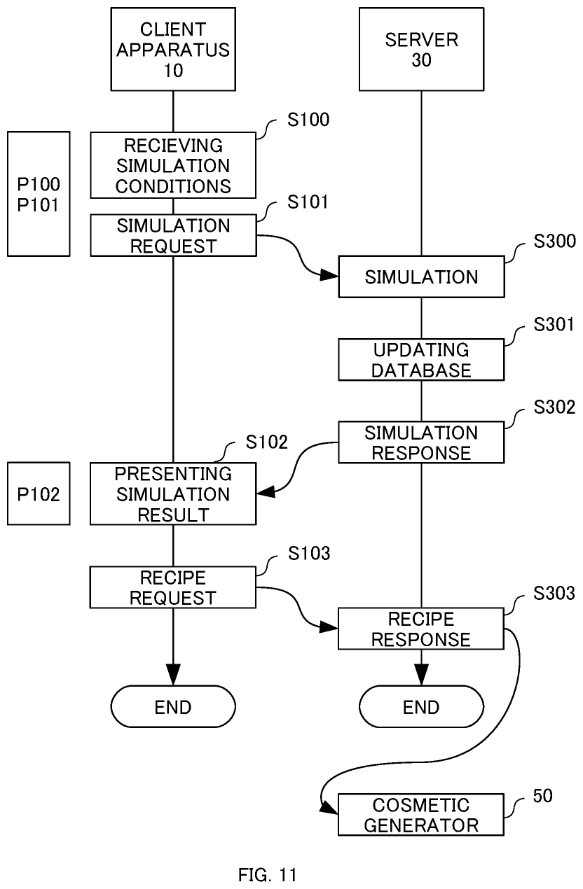

FIG. 11 is a sequence diagram of the simulation process of the present embodiment.

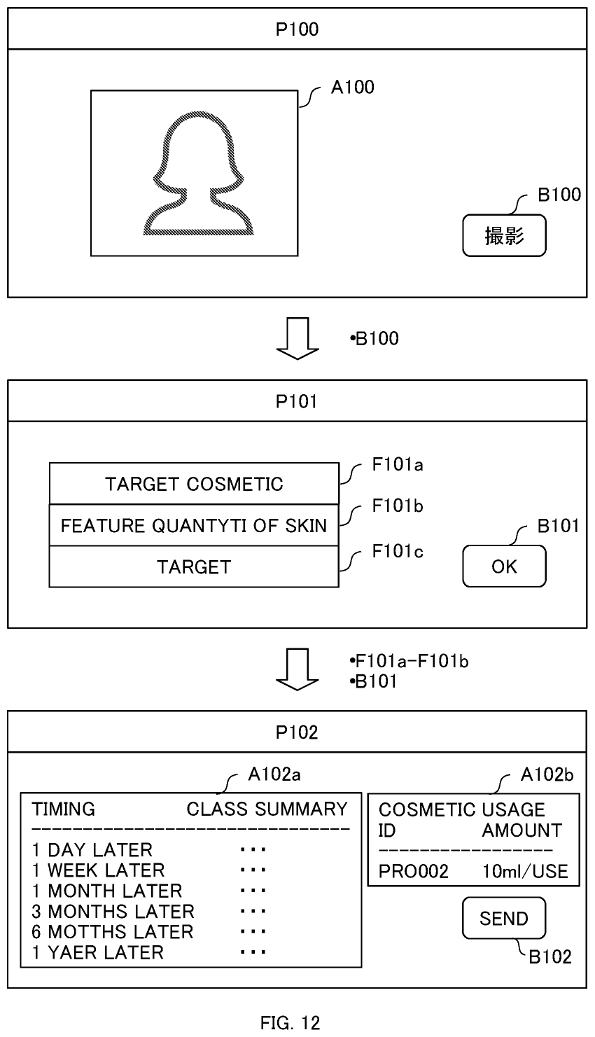

FIG. 12 is a diagram showing an example of a screen displayed in the information processing of FIG. 11.

As shown in FIG. 11, the client apparatus 10 executes receiving simulation conditions (S100).

Specifically, the processor 12 displays the screen P100 (FIG. 12) on the display.

The screen P100 includes a display object A100 and an operation object B100.

The display object A100 includes an image captured by a camera (not shown) of the client apparatus 10.

The operation object B100 is an object that receives a user instruction for executing capturing.

When a user (an example of a “simulation target person”) operates the operation object B100 when the display object A100 includes an image of the user's skin (for example, an image of the user's facial), the processor 12 generates image data (an example of “subject skin information”) of the image included in the display object A100.

The processor 12 displays the screen P101 (FIG. 12) on the display.

The screen P101 includes an operation object B101b and field objects F101a to F101c.

The field object F101a is an object that receives a user instruction for designating a target cosmetic (for example, a cosmetic used by the user) to be simulated.

The field object F101b is an object that receives a user instruction for designating the user's skin feature quantity (an example of “target person skin information”).

The user's skin feature quantity is, for example, at least one of water content, pores, texture, sebum amount, state of stratum corneum, and skin color.

The field object F101c is an object that receives a user instruction for designating a target to be simulated.

After the step S100, the client apparatus 10 executes simulation request (S101).

Specifically, when the user inputs a user instruction to the field objects F101a to F101b and operates the operation object B101b, the processor 12 transmits simulation request data to the server 30.

The simulation request data includes the following information:

user ID of the simulation target person (hereinafter referred to as “target user ID”);

image data generated in step S100;

target cosmetic ID of the target cosmetic corresponding to the user's instruction given on the field object F101a; and

feature quantity given on the field object F101b.

After the step S101, the server 30 executes simulation (S300).

Specifically, the processor 32 extracts the feature quantity from the image data included in the simulation request data.

The processor 32 identifies the skin characters of the user based on at least one of the feature quantity extracted from the image data and the feature quantity included in the simulation request data.

The processor 32 refers to the “skin character” field of the class information master database to specify the class ID associated with the skin character having the highest degree of similarity to the specified skin character.

The specified class ID indicates the class to which the skin corresponding to the image data included in the simulation request data belongs.

The processor 32 refers to the “class summary” field associated with the specified class ID to identify information regarding a summary description of the class to which the skin belongs.

The processor 32 refers to the “recommendation” field associated with the specified class ID to identify the recommendation information corresponding to the class to which the skin belongs.

The processor 32 refers to each subfield of the “transition probability” field of the class information master database, and specifies the transition probability for each subfield.

The specified transition probabilities are the class to which the skin belongs after 1 day, the class to which the skin belongs after 1 week, the class to which the skin condition after 1 month belongs, the class to which the skin condition belongs after 3 months, the class to which the skin condition belongs after 6 months, and the class to which the skin condition belongs after one year.

After the step S300, the server 30 executes updating database (S301).

Specifically, the processor 32 adds a new record to the simulation log information database (FIG. 10) associated with the target user ID included in the simulation request data.

The following information is stored in each field of the new record:

in the “simulation log ID” field, new simulation ID is stored;

in the “date and time” field, information regarding the execution date and time of step S300 is stored;

in the “cosmetic ID” field, target cosmetic ID included in the simulation request data is stored;

in the “skin image” filed, image data included in the simulation request data is stored;

in the “1 day later” field of the “simulation result” field, the class ID of the class to which the skin after 1 day belongs is stored;

in the “1 week later” field of the “simulation result” field, the class ID of the class to which the skin after 1 week belongs is stored;

in the “1 month later” field of the “simulation result” field, the class ID of the class to which the skin after 1 month belongs is stored;

in the “3 months later” field of the “simulation result” field, the class ID of the class to which the skin after 3 months belongs is stored;

in the “6 months later” field of the “simulation result” field, the class ID of the class to which the skin after 6 months belongs is stored; and

in the “1 year later” field of the “simulation result” field, the class ID of the class to which the skin 1 year later belongs is stored.

After the step S301, the server 30 executes a simulation response (S302).

Specifically, the processor 32 transmits simulation response data to the client apparatus 10.

The simulation response data includes the following information:

class ID of the class to which the skin belongs one day later;

class ID of the class to which the skin belongs after one week;

class ID of the class to which the skin belongs after 1 month;

class ID of the class to which the skin belongs after 3 months;

class ID of the class to which the skin belongs after 6 months;

class ID of the class to which the skin belongs one year later;

the information regarding a class summary description specified in the step S300; and

the recommendation information specified in the step S300.

After the step S302, the client apparatus 10 executes presenting the simulation result (S102).

Specifically, the processor 12 displays the screen P102 (FIG. 12) on the display based on the simulation response data.

The screen P102 includes display objects A102a to A102b and an operation object B102.

The display object A102a includes class summary description of the class to which the skin belongs after 1 day, an class summary description of the class to which the skin belongs after 1 week, an class summary description of the class to which the skin belongs after 1 month, class summary description of the class to which the skin belongs after 3 months, class summary description of the class to which the skin belongs after 1 week, and class summary description of the class to which the skin belongs after one year.

The display object A102b includes the recommendation information suitable for the skin corresponding to the image data retrieved in the step S100.

The recommendation information is, for example, recommended cosmetic information regarding recommended cosmetic (for example, the cosmetic ID and usage amount of the recommended cosmetic).

The operation object B102 is an object that receives a user instruction for transmitting the recipe information of the recommended cosmetic to the cosmetic generator 50.

After the step S102, the client apparatus 10 executes the recipe request (S103).

Specifically, when the user operates the operation object B102, the processor 12 transmits the recipe request data to the server 30.

The recipe request data includes the cosmetic ID of the recommended cosmetic.

After the step S103, the server 30 executes the recipe response (S303).

Specifically, the processor 32 refers to the makeup master database (FIG. 6) and specifies the information (ingredient name and content ratio) in the “ingredient” field associated with the cosmetic ID included in the recipe request data. To do.

The processor 32 transmits the recipe information to the cosmetic generator 50.

The recipe information includes the following information:

information in the specified “ingredient” field; and

information on the usage amount of recommended cosmetic.

The cosmetic generator 50 generates cosmetics based on the recipe information transmitted from the server 30.

Specifically, the processor 52 determines the total extraction amount of the raw materials to be extracted from the plurality of cartridges 55a to 55b based on the information regarding the usage amount included in the recipe information.

The processor 52 determines the respective extraction amounts of the raw materials contained in the plurality of cartridges 55a to 55b based on the determined total extraction amount and the component names, and content ratios contained included in the recipe information.

The processor 52 generates a control signal for extracting each raw material contained in the plurality of cartridges 55a to 55b according to the determined extraction amount.

The extraction controller 56 extracts the raw materials contained in the plurality of cartridges 55a to 55b based on the control signal generated by the processor 52.

As a result, an appropriate amount of cosmetics recommended for utilization on the skin corresponding to the image data received in step S100 is generated.

According to the present embodiment, by giving an image of the skin to the client apparatus 10, the user can know the future skin condition when continuously using the cosmetic through the display object A102a, the recommended cosmetic through the display object A102b, and can obtain an appropriate amount of recommended cosmetic through the cosmetic generator 50.

### (5) Variation of Present Embodiment

Variations of the present embodiment are described.

### (5-1) First Variation

First variation of the present embodiment will be described.

First variation is an example of designating the target skin condition of the simulation target person.

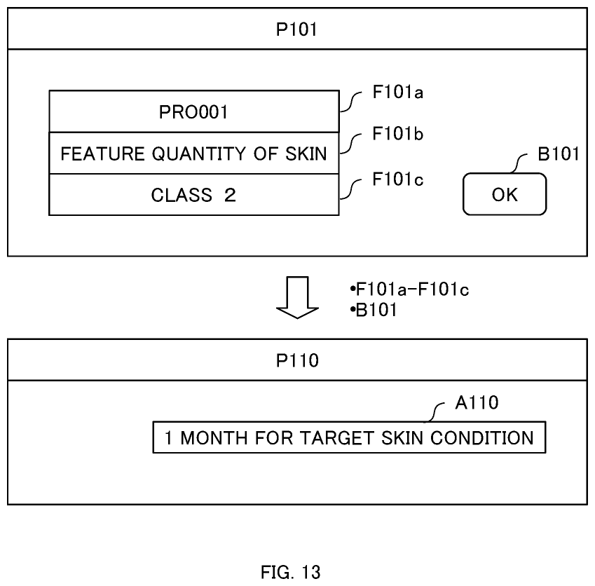

FIG. 13 is a view showing an example of a screen displayed in the information processing of the first variation.

In the first variation, in the step S100, the user designates the target cosmetics in the field object F101a and the target (as an example, the target skin condition) in the field object F101c.

In this case, the target specified in the field object F101c is assigned a class ID (an example of “target information”) corresponding to the target skin condition.

In the step S101, the processor 12 transmits the simulation request data to the server 30.

The simulation request data includes the following information:

target user ID;

the image data generated in the step S100;

the target cosmetic ID of the target cosmetic corresponding to the user instruction given in the field object F101a;

the feature quantity corresponding to the user instruction given in the field object F101b; and

class ID assigned to the target given to the field object F101c (hereinafter referred to as “target class ID”)

In the step S300, the processor 32 inputs the image data included in the simulation request data with respect to the condition transition model (FIG. 8) corresponding to the class information master database (FIG. 7) associated with the target cosmetic ID included in the simulation request data.

The processor 32 extracts the feature quantity from the image data.

The processor 32 specifies the skin characters of the user based on at least one of the feature quantity extracted from the image data and the feature quantity included in the simulation request data.

The processor 32 refers to the “skin character” field of the class information master database to specify the class ID associated with the skin character having the highest degree of similarity to the specified skin character.

The specified class ID indicates the class to which the skin corresponding to the image data included in the simulation request data belongs.

The processor 32 refers to the transition probability stored in the “transition probability” field associated with the specified class ID, and specifies the lead time required until the transition probability becomes equal to or higher than a predetermined transition probability.

For example, in the case that the class of skin condition corresponding to the image data included in the simulation data is class 1, the class of target skin condition is class 2, and the transition probability P12 equal to or higher than the predetermined transition probability is stored in the “1 month later” field, the processor 32 determines that the lead time required to reach the target skin condition is “1 month”.

After the step S301, in the step S302, the processor 32 transmits the simulation response data to the client apparatus 10.

The simulation response data includes information regarding the lead time determined in step S300.

After the step S302, in the step S102, the processor 12 displays the screen P110 (FIG. 13) on the display.

The screen P110 includes a display object A110.

The display object A110 includes the lead time to reach the target skin condition.

According to the first variation, by giving an image of the skin and the target skin condition to the client apparatus 10, the user can know the lead time to reach the target skin condition through the display object A110.

### (5-2) Second Variation

Second variation of the present embodiment will be described.

Second variation is an example of a method of generating a class information master database.

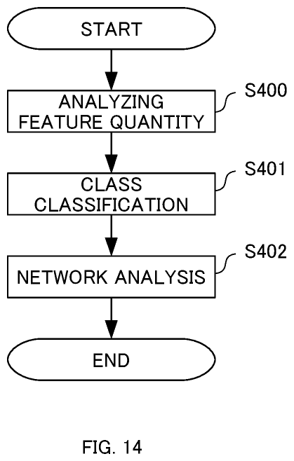

FIG. 14 is a flowchart of information processing of the second variation.

As shown in FIG. 14, the server 30 executes analyzing feature quantity (S400).

Specifically, the processor 32 extracts the skin feature quantity from the image data in the “skin image” field of the subject information database (FIG. 5).

The skin feature quantity is, for example, at least one of the amount of water, the pores, the texture, the amount of sebum, the state of the stratum corneum, and the skin color.

After the step S400, the server 30 executes class classification (S401).

The memory 31 stores a classification model.

The classification model is a trained model generated using deep learning.

The classification model defines the correlation between the skin feature quantity and class.

The processor 32 inputs the feature quantity obtained in the step S400 into the classification model to determine the class ID corresponding to the skin feature quantity of each skin image data.

The determined class ID is assigned to the feature quantity.

As a result, the class to which the skin condition determined from the feature quantity belongs is determined.

After the step S401, the server 30 executes network analysis (S402).

Specifically, the processor 32 refers to the subject information database (FIG. 5) to analyze a class of feature quantity of a plurality of skin image data associated with one subject ID, and specify the transition of the class to which the subject's skin belongs.

The processor 32 refers to the transition of all subject ID classes to calculate statistical value of the transition probability for the transition to the class to which the skin belongs after 1 day, to the class to which the skin belongs after 1 week, to the class to which the skin condition after 1 month belongs, and to the class to which the skin condition after 3 months, the class to which the skin condition after 6 months, and the class to which the skin condition after one year.

The processor 32 stores the calculated transition probability in the “transition probability” field of the class information master database (FIG. 7).

### (5-3) Third Variation

The third variation is an example of creating a community of multiple classes.

### (5-3-1) Summary of Third Variation

The summary of the third variation will be described.

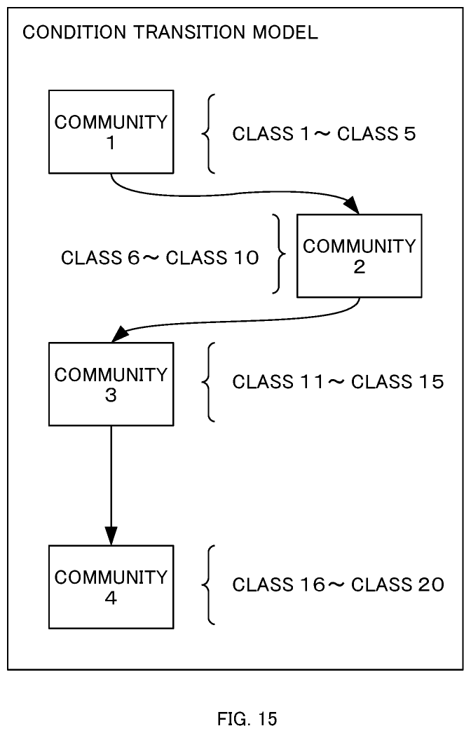

FIG. 15 is an explanatory diagram of summary of the third variation.

As shown in FIG. 15, a community is assigned to each class of the third variation.

For example, classes 1 to 5 are assigned to community 1.

Classes 6 to 10 are assigned to community 2.

Classes 11 to 15 are assigned to community 3.

Classes 16-20 are assigned to community 4.

This means that:

the tendency of changes in skin characters of classes 1 to 5 is common to each other;

the tendency of changes in skin characters of classes 6 to 10 is common to each other;

the tendency of changes in skin characters of classes 11 to 15 is common to each other;

the tendency of changes in skin characters of classes 16 to 20 is common to each other;

the class belonging to community 1 is likely to transition to a class belonging to an adjacent community (for example, community 2);

the class belonging to community 2 is likely to transition to a class belonging to an adjacent community (for example, community 1 or community 3); and

the class belonging to community 3 is likely to transition to a class belonging to an adjacent community (for example, community 2 or community 4).

That is, the condition transition model of the third variation defines the probability of transition between communities.

In other words, this condition transition model defines the transition of the tendency of changes in skin characters.

(5-3-2) Community information database

The community information database of the third variation will be described.

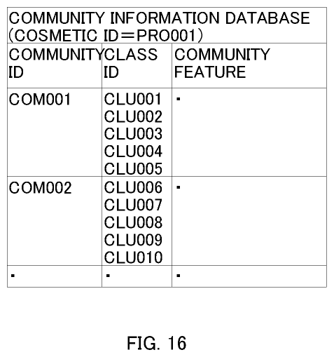

FIG. 16 is a diagram showing a data structure of the community information database of the third variation.

Community information is stored in the community information database of FIG. 16.

Community information is information regarding a community composed of a plurality of classes having common characteristics.

The community information database includes a “community ID” field, a “class ID” field, and a “community feature” field.

Each field is associated with each other.

The community information database is associated with the cosmetic ID.

The “community ID” field stores community ID.

The community ID is an example of community identification information that identifies a community.

The “class ID” field stores the class ID of the class assigned to the community.

The “community feature” field stores information regarding community features (hereinafter referred to as “community feature information”).

The community feature information includes, for example, at least one of the following information:

information on skin groove uniformity:

information on skin groove area;

information on skin groove irradiance;

information on skin groove definition; and

information on pore size.

### (5-3-3) Information Processing

The information processing of the third variation will be described.

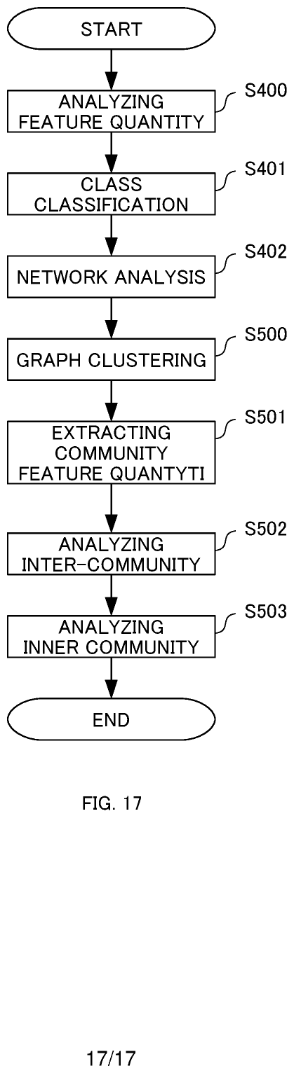

FIG. 17 is a sequence diagram of the processing of the community analysis of the third variation.

As shown in FIG. 17, the server 30 executes graph clustering (S500) after the steps S400 to S402 as in the second variation.

Specifically, the processor 32 applies the following method with respect to the information in the “skin image” field and the “skin condition” field (that is, the combination of the skin image data and the skin condition information) of the subject information master database (FIG. 5) to extract the community of each class by applying either method.

It means that transitions between skin classes belonging to the same community are easy (that is, the transition probability between classes is high).

Modularity Q maximization method;

method using greedy algorithm; and

method using edge mediation.

The processor 32 assigns a unique community ID to the extracted community.

The processor 32 stores the community ID assigned to each community in the “community ID” field of the community information database (FIG. 16).

The processor 32 stores the class ID of the class to which each community is assigned in the “class ID” field.

After the step S500, the server 30 executes analyzing inter-community (S501).

Specifically, the processor 32 extracts the feature quantity of the skin image data (hereinafter referred to as “community feature quantity”) corresponding to the class assigned to the community for each community.

Community features include, for example, following:

skin groove uniformity;

skin groove area;

skin groove irradiance;

skin groove definition; and

pore size.

The processor 32 normalize the extracted community feature quantity to calculate the statistical value of the feature quantity of each community.

The processor 32 stores the statistical value of each community feature quantity in each subfield of the “community feature” field of the community information database (FIG. 16).

As a result, as shown in FIG. 16, a transition path between communities is obtained.

After the step S501, the server 30 executes analyzing the inner community (S502).

Specifically, the processor 32 calculates a statistical value of community features (hereinafter referred to as “community features”) for each class belonging to each community.

The processor 32 specifies the features (specifically, changes in the community features) between the classes constituting each community in each community based on the features in the community.

According to the third variation, the main transition of the user's skin can be identified.

### (6) Summary of the Present Embodiment

This embodiment is summarized.

The first aspect of the present embodiment is

an information processing apparatus that executes a simulation that predicts future skin condition when cosmetics are used on the skin, the apparatus:

retrieving (for example, the processor 32 that executes the step S300) target person skin information regarding simulation target person's skin;

predicting (for example, the processor 32 that executes the step S300) transition of skin condition of the simulation target person by inputting the target person's skin information to condition transition model relating to the skin condition transition based on subject skin information indicating the time course of the skin when each of the plurality of subjects uses the cosmetic on the skin, subject condition information regarding the subject's skin condition corresponding to each subject's skin information, and cosmetic information regarding the cosmetic; and

presenting (for example, the processor 32 that executes the step S302) the predicted skin condition transition.

According to the first aspect, by inputting the target person skin information into the condition transition model based on subject skin information of the plurality of subjects, the subject condition information of the plurality of subjects, and the cosmetics information, the transition of the skin condition of the simulated subject is predicted.

It may predict the future skin condition when continuously using cosmetics.

The second aspect of the present embodiment is that the apparatus

retrieves (for example, the processor 32 that executes the step S300) target information regarding target skin condition of the simulation target person, and

predicts lead time required for the skin condition of the simulation target person to reach the skin condition corresponding to the target information.

According to the second aspect, the lead time to reach the target skin condition of the simulation target person is predicted.

It may motivate the simulation target person to continue the care behavior until the target skin condition is reached.

The third aspect of the present embodiment is that the apparatus presents (for example, the processor 32 that executes the step S302) recommendation information regarding skin care or makeup suitable for the predicted skin condition.

According to the third aspect, the recommendation information according to the prediction result of the skin condition is presented.

It may guide the simulation target person to appropriate skin care or makeup.

The fourth aspect of the present embodiment is that the apparatus

retrieves (for example, the processor 32 that executes the step S300) cosmetic information regarding the cosmetic used on the skin by the simulation target person, and

presents (for example, the processor 32 that executes the step S302) the recommendation information regarding a method of using the cosmetic based on the combination of the predicted skin condition and the cosmetic information.

According to the fourth aspect, the recommendation information regarding the usage method of the cosmetics in utilization is presented according to the prediction result of the skin condition.

It may guide the simulation target person to appropriate skin care or makeup.

A fifth aspect of the present embodiment is that the apparatus presents recommended cosmetic information regarding recommended cosmetic recommended for utilization according to the predicted skin condition.

According to the fifth aspect, information on recommended cosmetic according to the prediction result of the skin condition is presented.

It may guide the simulation target person to appropriate skin care or makeup.

The sixth aspect of the present embodiment is that the recommended cosmetic information includes information on usage amount of the recommended cosmetic.

According to the sixth aspect, information on the usage amount of recommended cosmetic according to the prediction result of the skin condition is presented.

It may guide the simulation target person to appropriate skin care or makeup.

The seventh aspect of the present embodiment is that the apparatus transmits (for example, the processor 32 that executes the step S303) recipe information for generating the recommended cosmetic to cosmetic generator for generating the cosmetics.

According to the seventh aspect, the recipe information of the recommended makeup according to the prediction result of the skin condition is transmitted to the cosmetic generator 50.

It may make the simulation target person to easily get the recommended cosmetic.

The eighth aspect of the present embodiment is that the apparatus

retrieves (for example, the processor 32 that executes the step S300) cosmetic information regarding target cosmetics of the simulation, and

predicts the transition of the skin condition of the simulation target person by inputting the cosmetic information of the target cosmetic and the skin information of the target person into the condition transition model.

According to the eighth aspect, it may predict the future skin condition when the user continuously uses the target cosmetics arbitrarily designated.

The ninth aspect of the present embodiment is that the condition transition model (for example, FIG. 8) defines a class corresponding to each skin condition and a link between a plurality of classes.

The tenth aspect of the present embodiment is that the condition transition model (for example, FIG. 15) includes multiple communities to which skin conditions have a common tendency to change skin features, and defines transitions between communities.

According to the tenth aspect, it may specify the main transition of the user's skin.

The eleventh aspect of the present embodiment is a cosmetic generator 50 that can be connected to the information processing apparatus (for example, the server 30) of, the generator comprising:

multiple cartridges 55a to 55b configured to contain cosmetic ingredients, wherein

the generator determines (for example, the processor 52) extraction amount of raw material contained in each cartridge 55a to 55b based on the recipe information transmitted by the information processing apparatus, and

extracts (for example, the processor 52) the raw materials contained in each cartridge based on the determined extraction amount.

According to the eleventh aspect, the recipe information of the recommended makeup according to the prediction result of the skin condition is transmitted to the cosmetic generator 50.

It may make the simulation target person to easily get the recommended cosmetic.

The twelfth aspect of the present embodiment is that

when the recipe information includes information on the usage amount of recommended cosmetic according to the predicted skin condition, the generator 50 determines the extraction amount so that total extraction amount of the raw materials contained in the plurality of cartridges is equal to the usage amount.

According to the twelfth aspect, the recipe information of the recommended makeup according to the prediction result of the skin condition is transmitted to the cosmetic generator 50.

It may make the simulation target person to easily get the recommended cosmetic.

A thirteenth aspect of the present embodiment is a computer program for causing a computer (for example, a processor 32) to function as each of the means described in any of the above.

### (7) Other Variations

Other Variations will be described.

The memory 11 may be connected to the client apparatus 10 via the network NW.

The memory 31 may be connected to the server 30 via the network NW.

Each step of the above information processing can be executed by either the client apparatus 10 or the server 30.

In the above embodiment, an example of retrieve the feature quantity of the user's skin based on the user's instruction is shown.

However, this embodiment can also be applied to the case where the feature quantity is stored from the moisture measuring apparatus.

Although the embodiments of the present invention are described in detail above, the scope of the present invention is not limited to the above embodiments.

Further, various modifications and changes can be made to the above embodiments without departing from the spirit of the present invention.

In addition, the above embodiments and variations can be combined.

### REFERENCE SIGNS LIST

1: Information processing system
10: Client apparatus
11: Memory
12: Processor
13: Input and output interface
14: Communication interface
30: Server
31: Memory
32: Processor
33: Input and output interface
34: Communication interface
50: Cosmetic generator
51: Memory
52: Processor
53: Input and output interface
54: Communication interface
55a, 55b: Cartridge
56: Extraction controller

1. An information processing apparatus that executes a simulation that predicts future skin condition when cosmetics are used on the skin, the apparatus:
retrieving target person skin information regarding simulation target person's skin;
predicting transition of skin condition of the simulation target person by inputting the target person's skin information to condition transition model relating to the skin condition transition based on subject skin information indicating the time course of the skin when each of the plurality of subjects uses the cosmetic on the skin, subject condition information regarding the subject's skin condition corresponding to each subject's skin information, and cosmetic information regarding the cosmetic; and
presenting the predicted skin condition transition.

2. The apparatus of claim 1, wherein the apparatus retrieves target information regarding target skin condition of the simulation target person, and
predicts lead time required for the skin condition of the simulation target person to reach the skin condition corresponding to the target information.

3. The apparatus of claim 1, wherein the apparatus presents recommendation information regarding skin care or makeup suitable for the predicted skin condition.

4. The apparatus of claim 3, wherein the apparatus retrieves cosmetic information regarding the cosmetic used on the skin by the simulation target person, and
presents the recommendation information regarding a method of using the cosmetic based on the combination of the predicted skin condition and the cosmetic information.

5. The apparatus of claim 1, the apparatus presents recommended cosmetic information regarding recommended cosmetic recommended for utilization according to the predicted skin condition.

6. The apparatus of claim 5, wherein the recommended cosmetic information includes information on usage amount of the recommended cosmetic.

7. The apparatus of claim 5, wherein the apparatus transmits recipe information for generating the recommended cosmetic to cosmetic generator for generating the cosmetics.

8. The apparatus of claim 1, wherein the apparatus retrieves cosmetic information regarding target cosmetics of the simulation, and
predicts the transition of the skin condition of the simulation target person by inputting the cosmetic information of the target cosmetic and the skin information of the target person into the condition transition model.

9. The apparatus of claim 1, wherein the condition transition model defines a class corresponding to each skin condition and a link between a plurality of classes.

10. The apparatus of claim 9, wherein the condition transition model includes multiple communities to which skin conditions have a common tendency to change skin features, and defines transitions between communities.

11. A cosmetic generator that can be connected to the information processing apparatus of claim 1, the generator comprising:
multiple cartridges configured to contain cosmetic ingredients, wherein
the generator determines extraction amount of raw material contained in each cartridge based on the recipe information transmitted by the information processing apparatus, and
extracts the raw materials contained in each cartridge based on the determined extraction amount.

12. The generator of claim 11, wherein when the recipe information includes information on the usage amount of recommended cosmetic according to the predicted skin condition, the generator determines the extraction amount so that total extraction amount of the raw materials contained in the plurality of cartridges is equal to the usage amount.

13. (canceled)

14. The apparatus of claim 2, wherein the apparatus presents recommendation information regarding skin care or makeup suitable for the predicted skin condition.

15. The apparatus of claim 14, wherein the apparatus retrieves cosmetic information regarding the cosmetic used on the skin by the simulation target person, and
presents the recommendation information regarding a method of using the cosmetic based on the combination of the predicted skin condition and the cosmetic information.

16. The apparatus of claim 2, the apparatus presents recommended cosmetic information regarding recommended cosmetic recommended for utilization according to the predicted skin condition.

17. The apparatus of claim 16, wherein the recommended cosmetic information includes information on usage amount of the recommended cosmetic.

18. The apparatus of claim 17, wherein the apparatus transmits recipe information for generating the recommended cosmetic to cosmetic generator for generating the cosmetics.

19. The apparatus of claim 2, wherein the apparatus retrieves cosmetic information regarding target cosmetics of the simulation, and
predicts the transition of the skin condition of the simulation target person by inputting the cosmetic information of the target cosmetic and the skin information of the target person into the condition transition model.

20. The apparatus of claim 2, wherein the condition transition model defines a class corresponding to each skin condition and a link between a plurality of classes.

21. A computer implemented method for a simulation that predicts future skin condition when cosmetics are used on the skin, the method comprising:
retrieving target person skin information regarding simulation target person's skin;
predicting transition of skin condition of the simulation target person by inputting the target person's skin information to condition transition model relating to the skin condition transition based on subject skin information indicating the time course of the skin when each of the plurality of subjects uses the cosmetic on the skin, subject condition information regarding the subject's skin condition corresponding to each subject's skin information, and cosmetic information regarding the cosmetic; and
presenting the predicted skin condition transition.
